(19) United States
(12) Patent Application Publication
(10) Pub. No.: US 2016/0206009 A1
(43) Pub. Date: Jul. 21, 2016

(54) Apparatus for Tucking Genitalia

(71) Applicant: William Claney, Clayton, CA (US)

(72) Inventor: William Claney, Clayton, CA (US)

(21) Appl. No.: 14/996,929

(22) Filed: Feb. 9, 2016

Related U.S. Application Data
(60) Provisional application No. 62/103,980, filed on Jan. 15, 2015.

(51) Int. Cl. A41B 9/02; A61F 5/40

(52) U.S. Cl. CPC A41B 9/02; A61F 5/40

(57) ABSTRACT

An apparatus for tucking the penis in between the legs of the user contains a first penis ring, a first tether, a fastener, and a groove. A first end of the first tether is positioned within the groove. A body of the first tether extends from the first end to a second end which is attached to the fastener. The fastener is used to attach the first tether which is pulled in between the legs and along the glutes of the user. More specifically, the fastener is attached to an attachment band. The user adjusts the tension applied on the first tether by sliding the attachment band along an abdomen section of the user. A second penis ring can also be used along with the first penis ring.

The current application claims a priority to the U.S. Provisional Patent application Ser. No. 62/103,980 filed on Jan. 15, 2015.

### FIELD OF THE INVENTION

The present invention relates generally to the field of tucking genitalia. More specifically, the present invention is an apparatus which allows a user to tuck a penis between the legs.

### BACKGROUND OF THE INVENTION

The term ‘tucking’ generally refers to the process of tucking a penis and testicles back through the legs towards the buttocks and holding the tuck through friction. Tucking is generally done for sustainability purposes, appearance, and comfort.

Different methods are currently being used when tucking the penis. Different types of holders and adhesive are currently used for tucking. Even though the intended purpose is fulfilled for a limited time period, many disadvantages can also be seen with these current tucking methods. Discomfort, difficulty in walking, and difficulty in urinating are among some of the main difficulties. Additionally, when the legs are squeezed together undue pressure is applied on the testicles which can lead to harmful circumstances. Since the penis does not remain stationary with the existing tucking methods, an unwanted bulge can appear. The bulge can negatively affect the individual seeking to showcase a flat front appearance.

In addition to holders and adhesive, different types of clothing garments are also used to tuck the penis in between the legs of the user. However, when wearing these clothing items for an extended time period it can be stressful to the user. The additional clothing items can also have a negative effect on the aesthetically pleasing appearance of the user.

The objective of the present invention is to address the aforementioned issues. More specifically, the present invention allows the user to conveniently tuck the penis between the legs without any disturbance to activities such as walking or urinating. By utilizing the present invention the desired feminine look can be obtained conveniently. Moreover, the need to undergo surgeries and use different types of adhesive to hold the penis in between the legs is also eliminated by using the present invention. The present invention helps the user have a smooth non-bulge frontal presentation that emulates a female's vagina or labia major.

### BRIEF DESCRIPTION OF THE DRAWINGS

FIG. 1 is an illustration of the present invention, wherein a quick release clip is used as the fastener.

FIG. 2 is another illustration of the present invention, wherein a lobster claw clip is used as the fastener.

FIG. 3 is an illustration of the present invention, wherein a second penis ring is used in addition to the first penis ring.

FIG. 4 is a front view illustration of a user when the present invention is being used.

FIG. 5 is a rear view illustration of the user when the present invention is being used.

FIG. 6 is a side view illustration of the user, when both the first penis ring and the second penis ring is used.

FIG. 7A is a perspective view of the first penis ring.

FIG. 7B is a perspective view of the second penis ring.

FIG. 8 is a perspective view of the first penis ring with the flange.

FIG. 9 is an illustration of the present invention, wherein a second tether is used along with the first tether.

### DETAIL DESCRIPTIONS OF THE INVENTION

All illustrations of the drawings are for the purpose of describing selected versions of the present invention and are not intended to limit the scope of the present invention.

The present invention introduces an apparatus that allows a user to tuck a penis between the legs and have the penis stationary for an extended time period. By utilizing the present invention, the user can obtain the desired flat front appearance. In other words, a valley that appears to be a vagina and a labia major can be obtained by utilizing the present invention.

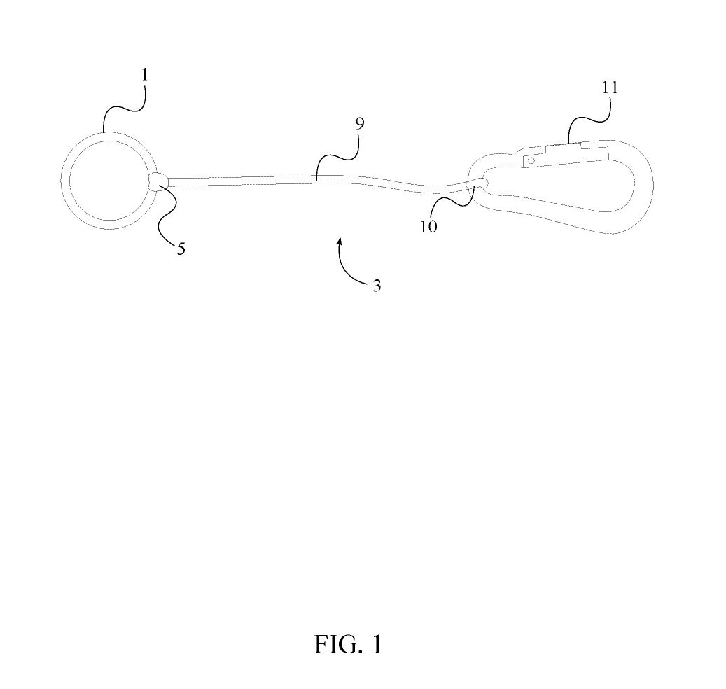

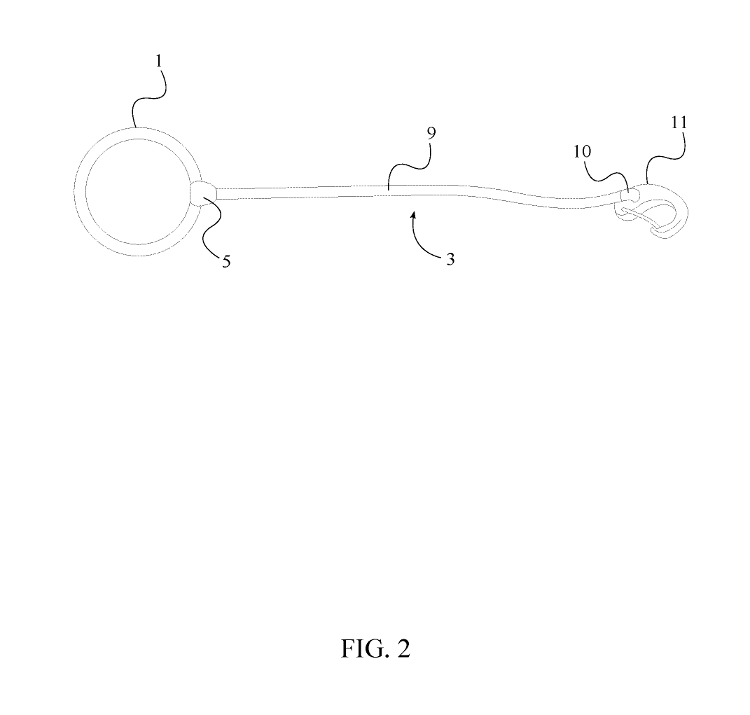

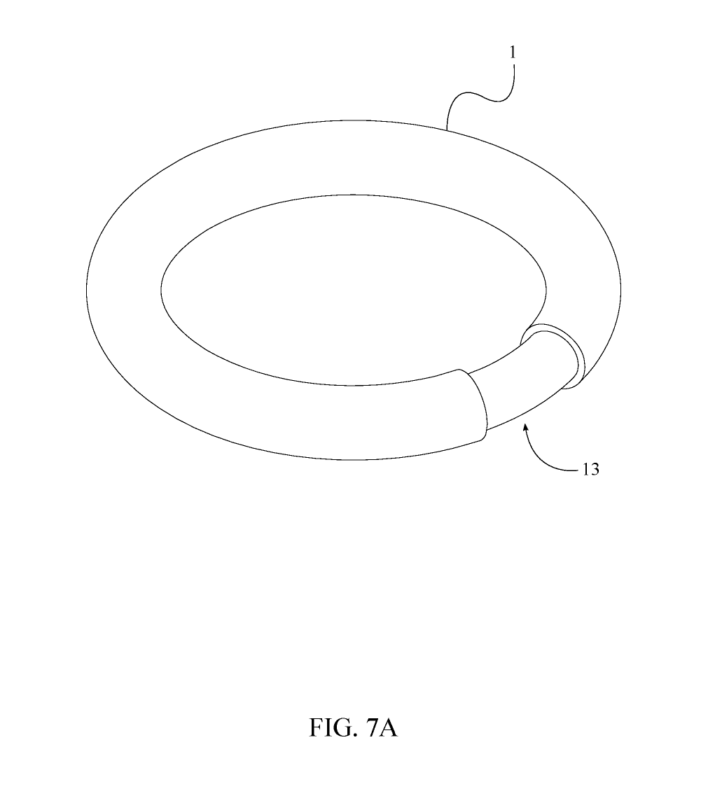

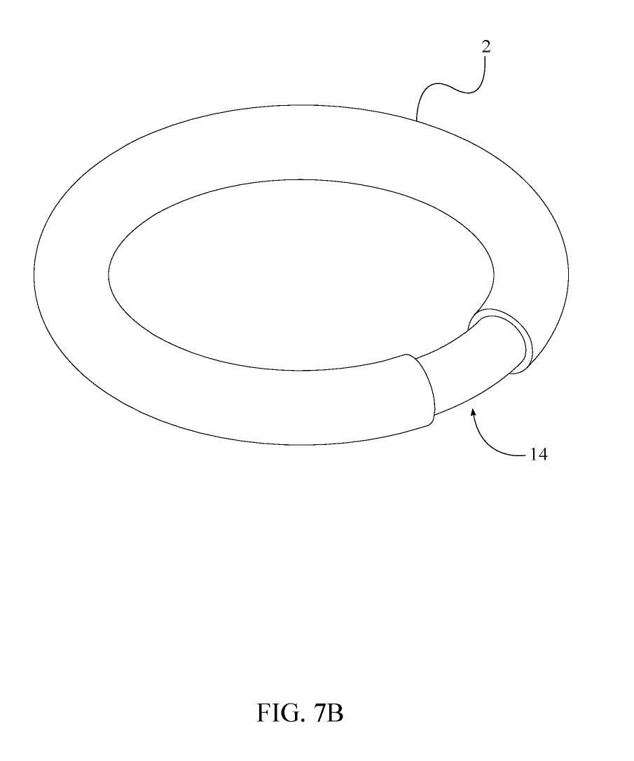

As illustrated in FIGS. 1-3, the present invention comprises a first penis ring 1, a first tether 3, a fastener 11, and a groove 13. The first penis ring 1 is appropriately sized to receive the penis of the user. In other words, when the present invention is being used, the user inserts the penis into the first penis ring 1. The first penis ring 1 is available in a variety of sizes for different sized penises. The first tether 3 comprises a first end 5, a body 9, and a second end 10 and is used for holding the penis stationary in between the legs of the user. The body 9 extends from the first end 5 to the second end 10. The first end 5 is positioned within the groove 13 which is radially positioned about the first penis ring 1 as seen in FIG. 7A. Since the first end 5 is positioned within the groove 13, the first end 5 lies flush against an inner surface of the first penis ring 1 eliminating a bulge that can occur due to the first end 5. Therefore, the user feels no discomfort when the penis is positioned within the first penis ring 1. The second end 10 of the first tether 3 is attached to the fastener 11. The fastener 11 is used to attach the first tether 3 onto an attachment band 15 worn by the user. The fastener 11 can vary in different embodiments of the present invention. As seen in FIG. 1 and FIG. 2, the fastener 11 can be, but is not limited to, a lobster claw clip or a quick release clip.

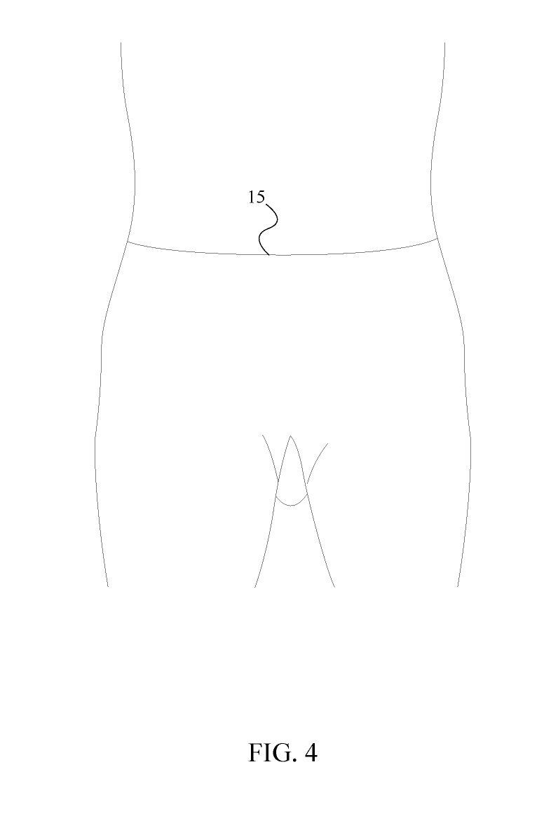

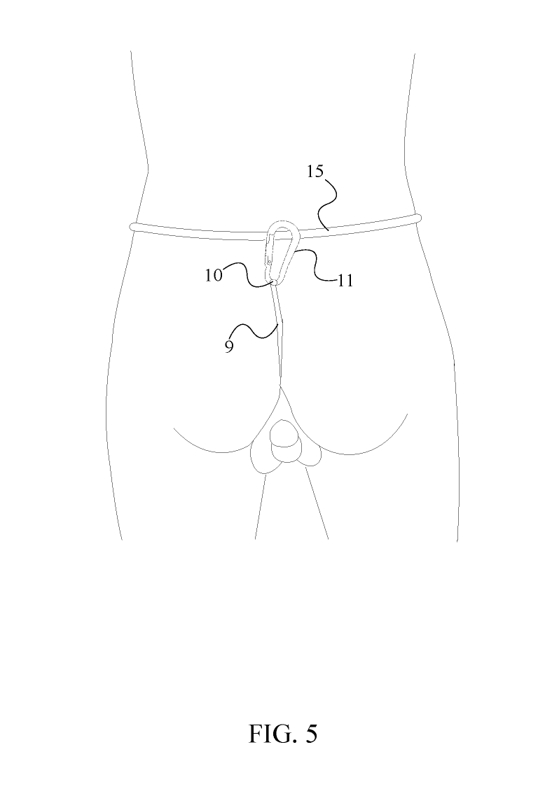

As seen in FIGS. 4-6, in the process of securing the penis in between the legs, the fastener 11 is removably attached to the attachment band 15. The attachment band 15 is size adjustable so that users with different waist sizes can use the present invention with equal convenience. In the preferred embodiment of the present invention, the attachment band 15 is a G-string. However, in another embodiment of the present invention the attachment band 15 can be a belt or other comparable item. In addition to attaching the fastener 11, the attachment band 15 is also used to determine the tension applied on the first tether 3. More specifically, if the user expects a tighter tuck by applying more tension on the first tether 3, the attachment band 15 is worn higher up on an abdomen section of the user. In contrast, if the user expects less tension on the penis and the first tether 3, the attachment band 15 is worn lower on the abdomen section. Therefore, by varying the position of the attachment band 15, the tension on the first tether 3 can be controlled, and thereby the tightness of the penis tuck can also be controlled.

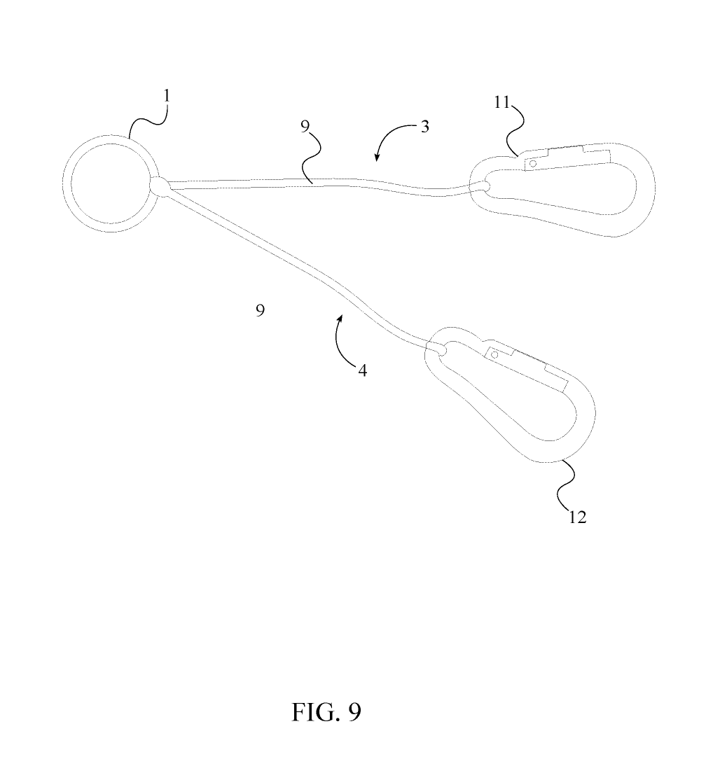

In another embodiment of the present invention, a second tether 4 can be used along with the first tether 3 as shown in FIG. 9. The second tether 4 is identical to the first tether 3. Therefore, the second tether 4 also comprises a first end 5, a body 9, and a second end 10. In the process of attaching the second tether 4 to the first penis ring 1, the first end 5 of the second tether 4 is also positioned within the groove 13. The second end 10 of the second tether 4 is attached to a second fastener 12. Similar to the fastener 11, the second fastener 12 is removably attached to the attachment band 15. Moreover, the second fastener 12 can be, but is not limited to, the lobster claw clip or the quick release clip. When only the first tether 3 is used, the first tether 3 is pulled along the glutes such that an anal sphincter of the user is covered. However, when both the first tether 3 and the second tether 4 are used, the anal sphincter is left uncovered since the first tether 3 and the second tether 4 are pulled only along the glutes and not in between the legs.

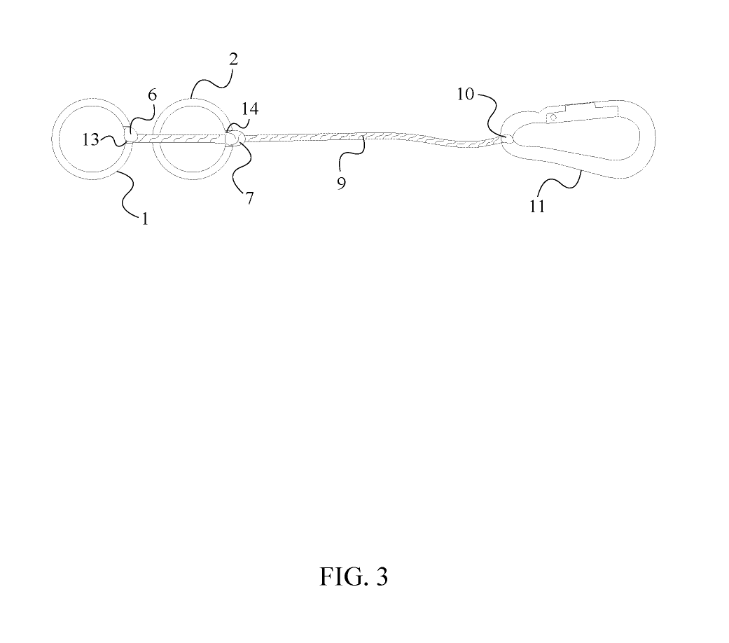

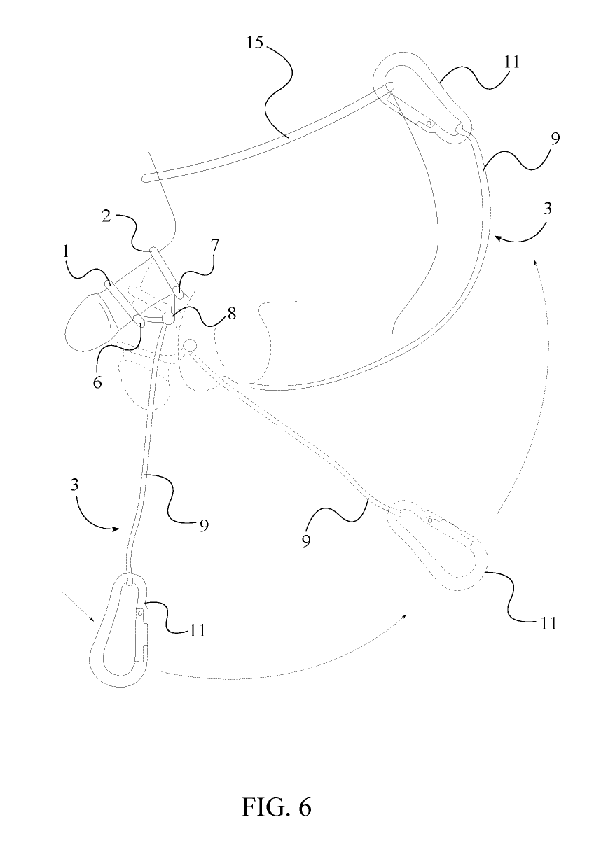

In the preferred embodiment of the present invention, the penis is inserted into the first penis ring 1. However, a second penis ring 2 can also be used along with the first penis ring 1 in another embodiment of the present invention. FIG. 3 and FIG. 6 illustrates the second penis ring 2 being used along with the first penis ring 1. The penis is inserted to the second penis ring 2 similar to the manner in which the penis is inserted into the first penis ring 1. The use of the second penis ring 2 eases the pressure applied on the first penis ring 1, thereby providing more comfort to the user. Moreover, the use of the second penis ring 2 allows the user to establish a better connection between the present invention and the penis. The first end 5 of the first tether 3 comprises a first node 6, a second node 7, and a third node 8 in order to connect to the first penis ring 1 and the second penis ring 2 simultaneously. The third node 8 is positioned in between the first node 6 and the second node 7 resulting in a Y-shape. The third node 8, which is positioned at a base of the Y-shape, is attached to the body 9 of the first tether 3. In the process of establishing a connection with the first penis ring 1, the first node 6 is attached to the first penis ring 1 such that the first node 6 is positioned within the groove 13 associated with the first penis ring 1. Likewise, the second node 7 is attached to the second penis ring 2. In doing so, the second node 7 is positioned within a second groove 14, which is radially positioned about the second penis ring 2 as seen in FIG. 7B.

As mentioned earlier, both the first penis ring 1 and the second penis ring 2 are made available in multiple sizes. However, in order to adjust to slight size variations of the penis, the first penis ring 1 and the second penis ring 2 are made of a flexible material. The flexible material allows the user to adjust the size such that the first penis ring 1 and the second penis ring 2 snugly fit around the penis. However, the material is also sufficiently rigid such that the overall circular shape of the first penis ring 1 and the second penis ring 2 is maintained.

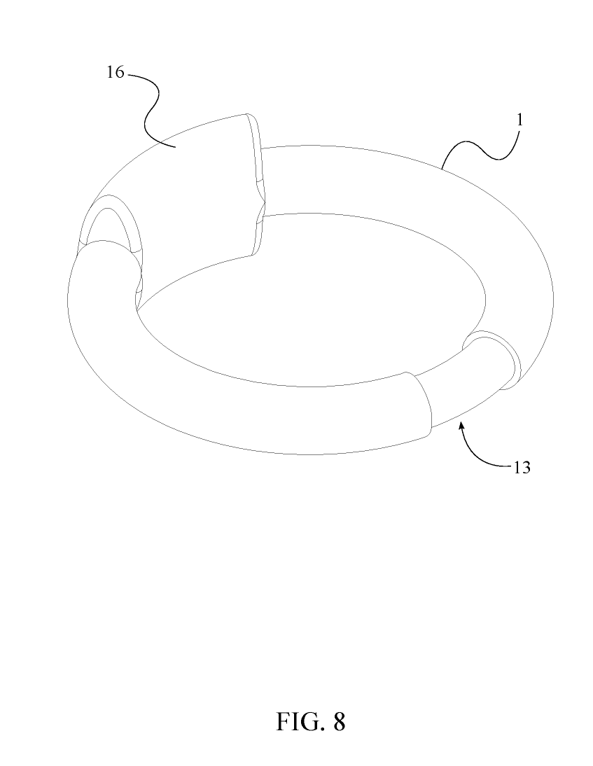

In order to provide comfort, the present invention comprises a flange 16 as seen in FIG. 8. When considering the first penis ring 1, the flange 16 is positioned along the first penis ring 1. The positioning of the flange 16 is such that the flange 16 is adjacently connected to the first penis ring 1 opposite the groove 13. The flange 16 on the second penis ring 2 is also similarly positioned. The extra surface area provided by the flange 16 provides additional comfort when the penis is tucked in between the legs of the user.

When utilizing the present invention, the following process flow can be followed. Initially, the attachment band 15 is worn around the waist. Next, the user inserts the penis into the first penis ring 1. If the second penis ring 2 is available, the penis is inserted into both the first penis ring 1 and the second penis ring 2. The first penis ring 1 is worn such that an external surface of the flange 16 is oriented upwards. When the first penis ring 1 is secured, the user grasps the fastener 11 at the second end 10 and the first tether 3 is pulled down as seen in FIG. 6. Next, the first tether 3 is pulled in between the legs and along the glutes of the user. Resultantly, the penis is tucked in between the legs of the user. When tucked, the external surface of the flange 16 is oriented towards the ground providing additional surface area for the penis to rest on. In order to secure the position of the penis, the fastener 11 is attached to the attachment band 15. The tension on the first tether 3 can be controlled by sliding the attachment band 15 along the abdomen section. If the second tether 4 is used in a different embodiment of the present invention, both the first tether 3 and the second tether 4 are pulled along the glutes allowing the anal sphincter to be available.

Although the invention has been explained in relation to its preferred embodiment, it is to be understood that many other possible modifications and variations can be made without departing from the spirit and scope of the invention as hereinafter claimed.

What is claimed is:

1. An apparatus for tucking genitalia between the legs comprises:
a first penis ring;
a first tether;
a fastener;
a groove;
the groove being radially positioned about the first penis ring;
the first tether comprises a first end, a body, and a second end;
the body extending from the first end to the second end;
the first end being positioned within the groove; and
the second end being attached to the fastener.

2. The apparatus for tucking genitalia between the legs as claimed in claim 1 comprises:
a second tether;
the second tether comprises a first end, a body, and a second end;
the first end of the second tether being positioned within the groove; and
the second end of the second tether being attached to a second fastener.

3. The apparatus for tucking genitalia between the legs as claimed in claim 1 comprises:
an attachment band; and
the fastener being removably attached to the attachment band.

4. The apparatus for tucking genitalia between the legs as claimed in claim 3, wherein the attachment band is size adjustable.

5. The apparatus for tucking genitalia between the legs as claimed in claim 3, wherein the attachment band is a G-string.

6. The apparatus for tucking genitalia between the legs as claimed in claim 3, wherein the attachment band is a belt.

7. The apparatus for tucking genitalia between the legs as claimed in claim 1 comprises:
the first end comprises a first node, a second node, and a third node;
the third node being positioned in between the first node and the second node; and
the third node being attached to the body.

8. The apparatus for tucking genitalia between the legs as claimed in claim 7 comprises:
the first node being attached to the first penis ring; and
the first node being positioned within the groove.

9. The apparatus for tucking genitalia between the legs as claimed in claim 1 comprises:
the second node being attached to a second penis ring;
the second node being positioned within a second groove; and
the second groove being radially positioned about the second penis ring.

10. The apparatus for tucking genitalia between the legs as claimed in claim 1, wherein the first penis ring is made of a flexible material.

11. The apparatus for tucking genitalia between the legs as claimed in claim 9, wherein the second penis ring is made of a flexible material.

12. The apparatus for tucking genitalia between the legs as claimed in claim 1, wherein the fastener is a lobster claw clip.

13. The apparatus for tucking genitalia between the legs as claimed in claim 1, wherein the fastener is a quick release clip.

14. The apparatus for tucking genitalia between the legs as claimed in claim 1 comprises:
a flange;
the flange being positioned along the first penis ring; and
the flange being adjacently connected to the first penis ring opposite the groove.
